SEQUENCE LISTING (2 sequences)

SEQ ID NO: 1 - protein, 6 aa, Artificial Sequence
  Apopep-1_linear form
CQRPPR

SEQ ID NO: 2 - protein, 7 aa, Artificial Sequence
  Apopep-1_cyclic form
CQRPPRC

(19) United States
(12) Patent Application Publication
(10) Pub. No.: US 2017/0340698 A1
(43) Pub. Date: Nov. 30, 2017

(54) CYCLIC PEPTIDE SPECIFICALLY BINDING TO APOPTOTIC CELLS AND USE THEREOF

(71) Applicant: KYUNGPOOK NATIONAL UNIVERSITY INDUSTRY-ACADEMIC COOPERATION FOUNDATION, Daegu (KR)

(72) Inventors: Byung-Heon LEE, Daegu (KR); In-San KIM, Daegu (KR); Hyun-Kyung JUNG, Daegu (KR)

(73) Assignee: KYUNGPOOK NATIONAL UNIVERSITY INDUSTRY-ACADEMIC CO OPERATION FOUNDATION, Daegu (KR)

(21) Appl. No.: 15/612,094

(22) Filed: Jun. 2, 2017

Related U.S. Application Data
(63) Continuation of application No. PCT/KR2015/013316, filed on Dec. 7, 2015.

(30) Foreign Application Priority Data
Dec. 5, 2014 (KR) 10-2014-0173936

(51) Int. Cl. A61K 38/12; C07K 7/52

(52) U.S. Cl. CPC A61K 38/12; C07K 7/52; A61K 2300/00

(57) ABSTRACT

Provided is a cyclic peptide (cyclo [Cys-Gln-Arg-Pro-Pro-Arg-Cys] peptide) comprised of the amino acid sequence of SEQ ID NO: 2; and a composition for apoptotic cell detection, drug delivery or imaging, containing the same as an active ingredient. The cyclic peptide (cyclo [Cys-Gln-Arg-Pro-Pro-Arg-Cys] peptide has an excellent effect of binding to apoptotic cells, compared with a linear peptide thereof, thereby greatly facilitating the detection of apoptotic cells and the in vivo imaging of an affected part under apoptosis, while the detection and imaging signal shows a very high correlation in disease prognosis prediction. The cyclic peptide binds to an imaging material, early diagnosing a response of a drug for treating diseases associated with abnormal cell proliferation, and binds to a therapeutic material, selectively delivering a drug to tissues afflicted with Apoptosis-associated diseases.

### TECHNICAL FIELD

The present application claims priority from and the benefit of Korean Patent Application No. 10-2014-0173936 filed on 5 Dec. 2014, which is hereby incorporated by reference for all purposes as if fully set forth herein.

The present invention relates to a cyclic peptide specifically binding to apoptotic cells and a use thereof and, more particularly, to a cyclic peptide consisting of the amino acid sequence of SEQ ID NO: 2 and a composition containing the same as an active ingredient for apoptotic cell detection, drug delivery, and imaging.

### BACKGROUND ART

Susceptibility to certain diseases and drug susceptibility to respective diseases differ from individual to individual. When prescribing a medication to a patient, a physician prescribes appropriate medicines according to his/her subjective judgment based on diagnostic results. In some cases, prescribed medicines may not show proper therapeutic effects, and thus, the physician may undergo trial and error of prescribing another medicine after evaluating the patient's progress. In these cases, it is important to promptly determine the patient's response (or susceptibility) to a medicine at an early stage and decide whether to continue using the same drug or replace it with another medicine. Especially in diseases needed for a long period of treatment, in order to reduce time and cost burden, it is very important to determine response and effects with respect to a particular treatment method or medicine early to determine the patient's treatment regimen.

Meanwhile, gastric cancer is the second leading cause of cancer death worldwide [1]. Single-agent chemotherapy for advanced gastric cancer uses capecitabine or 5-fluorouracil, while combined therapy (combination therapy) co-uses cisplatin and 5-fluorouracil, or cisplatin and capecitabine [2]. Unfortunately, gastric cancer has shown low response to chemotherapy. The response rate to chemotherapy in advanced gastric cancer ranges from 10 to 30% in single-agent therapy and from 30 to 60% in combination therapy, respectively [2]. In addition, molecular targeted drugs, such as cetuximab (anti-epidermal growth factor receptor antibody) and trastuzumab (anti-Her2 receptor antibody), have been used in combination with the chemotherapy to exhibit various response rates [3-5]. Considering these low response rates, it is very important to monitor and determine the response of stomach tumors early after treatment with an anticancer agent in view of the management of cancer treatment.

Traditionally, the determination of tumor response has been conducted by measuring the change of tumor size using computerized tomography (CT). However, with respect to the tumor response, the determination based on the tumor size can be ordinarily made only about two months after initiating drug treatment. According to the guidelines of the Response Evaluation Criteria in Solid Tumors (RECIST), when the size of the tumor is reduced by at least 30%, such a case is defined as having a partial response to the treatment [6], whereas when the size of the tumor is increased by 20% or more, such a case is considered as an ongoing disease. In order to reduce the time and cost required for tumor treatment, a means of determining earlier whether to continue the use of the same therapeutic method (or the same therapeutic substance) (go/no-go decision on the therapy) is required, instead of the conventional methods of determining the tumor response based on the tumor size through CT measurement.

The use of positron emission tomography (PET) imaging for measurement of ¹⁸F-fluorodeoxyglucose (¹⁸F-FDG) uptake by tumors is known to be a method capable of early determination of the tumor response after tumor treatment compared with a size-based CT imaging method. As the tumor cell mass and the tumor metabolism are reduced after chemotherapy, respectively, ¹⁸F-FDG uptake is reduced in the tumor tissue.

However, ¹⁸F-FDG uptake is known to be mainly dependent on the histopathological type of gastric cancer. For example, ¹⁸F-FDG uptake is low in signet-ring cell carcinoma and mucinous adenocarcinoma, which is due to the low level of GLUT-1 transporter [7, 8]. This feature restricts the determination of gastric cancer response by ¹⁸F-FDG uptake. Moreover, some types of tumors, such as breast cancer, show metabolic flare phenomenon (a temporary increase in ¹⁸F-FDG uptake after chemotherapy), which is difficult to discern from tumor relapse [9].

As described above, although it is very important to accurately determine (diagnose) early whether the therapeutic agent (and therapeutic method) exhibits the response in a subject having abnormal cell proliferation-related diseases, including cancer, there is a limitation that the current technique is difficult to apply widely because the detection effect is confined to only a disease of a specific region (or range). Therefore, there is a need for a means of being commonly utilized without being greatly influenced by specific mechanisms and histological characteristics of the diseases and accurately determining early the response to a used therapeutic substance (or therapeutic method).

Meanwhile, the inventors of the present invention have derived peptides capable of specifically targeting apoptotic cells that are undergoing apoptosis, through Korean Patents 10-0952841 and 10-1077618, and these peptides have been named ApoPep-1. The present inventors have verified in the above document that the ApoPep-1 linear peptides effectively target apoptosis that occurs in the affected tissues by apoptotic cell-related diseases, such as tumor diseases, neurodegenerative diseases, myocardial infarction, and arteriosclerosis.

However, in the practical use of these peptides, in order to allow the peptides to be used to image the affected portions and provide accurate diagnostic information, the fact that these peptides exhibit an increased detection rate due to characteristics based on the amino acid sequences of the peptides is not sufficient. That is, in the practical use of the peptides, there are various limiting factors in association with structure, stability, safety, dose, and effect, and in particular, a significant correlation between measured information and actual prognosis is required.

### PRIOR ART DOCUMENTS

### Patent Documents

Korean Patent Registration No. 10-0952841
Korean Patent Registration No. 10-1077618

### Non-Patent Documents

[1] Lozano R, Naghavi M, Foreman K, Lim S, Shibuya K, et al. (2012) Global and regional mortality from 235 causes of death for 20 age groups in 1990 and 2010: a systematic analysis for the Global Burden of Disease Study 2010. Lancet 380: 2095128.
[2] Sastre J, Garcia-Saenz J A, Diaz-Rubio E (2006) Chemotherapy for gastric cancer. World J Gastroenterol 12: 20413.
[3] Lordick F, Kang Y K, Chung H C, Salman P, Oh S C, et al. (2013) Capecitabine and cisplatin with or without cetuximab for patients with previously untreated advanced gastric cancer (EXPAND): a randomised, open-label phase 3 trial. Lancet Oncol 14: 49099.
[4] Bang Y J, Van Cutsem E, Feyereislova A, Chung H C, Shen L, et al. (2010) Trastuzumab in combination with chemotherapy versus chemotherapy alone for treatment of HER2-positive advanced gastric or gastro-oesophageal junction cancer (ToGA): a phase 3, open-label, randomised controlled trial. Lancet 376: 68797.
[5] Casadei R, Rega D, Pinto C, Monari F, Ricci C, et al. (2009) Treatment of advanced gastric cancer with cetuximab plus chemotherapy followed by surgery. Report of a case. Tumori 95: 81114.
[6] Padhani A R, Olivier L (2001) The RECIST (Response Evaluation Criteria in Solid Tumors) criteria: implications for diagnostic radiologists. Br J Radiol 74: 98386.
[7] Yoshioka T, Yamaguchi K, Kubota K, Saginoya T,
Yamazaki T, et al. (2003) Evaluation of 18F-FDG PET in patients with advanced, metastatic, or recurrent gastric cancer. J Nucl Med 44: 69099.
[8] Alakus H, Batur M, Schmidt M, Drebber U, Baldus S E, et al. (2010) Variable 18F-fluorodeoxyglucose uptake in gastric cancer is associated with different levels of GLUT-1 expression. Nucl Med Commun 31: 53238.
[9] Tu D G, Yao W J, Chang T W, Chiu N T, Chen Y H (2009) Flare phenomenon in positron emission tomography in a case of breast cancer pitfall of positron emission tomography imaging interpretation. Clin Imaging 33: 46870.

### DETAILED DESCRIPTION OF THE INVENTION

### Technical Problem

Therefore, the present inventors, while seeking a means of allowing an accurate direction design at an early stage in the prescription of a therapeutic method and a therapeutic substance for abnormal cell proliferation-related diseases including tumors, have verified that the cyclic peptide (cyclo[Cys-Gln-Arg-Pro-Pro-Arg-Cys] peptide) of the present invention has an excellent effect of accurately detecting the tumor response to a test preparation with high sensitivity at an early stage, compared with a linear peptide, and is closely correlated with tumor size reduction later, and therefore, the present inventors have completed the present invention.

Accordingly, an aspect of the present invention is to provide a cyclic peptide (cyclo[Cys-Gln-Arg-Pro-Pro-Arg-Cys] peptide) consisting of the amino acid sequence represented by SEQ ID NO: 2 and specifically binding to apoptotic cells, and to a use thereof.

Another aspect of the present invention is to provide a method for treating a neoplastic disease by administering an effective amount of the peptide and an anti-tumor substance conjugated thereto to a subject in need thereof.

Still another aspect of the present invention is to provide a method for preventing or treating a neurodegenerative disease by administering an effective amount of the peptide and a neurodegenerative disease therapeutic substance conjugated thereto to a subject in need thereof.

Still another aspect of the present invention is to provide a method for preventing or treating myocardial infarction by administering an effective amount of the peptide and a myocardial infarction therapeutic substance conjugated thereto to a subject in need thereof.

Still another aspect of the present invention is to provide a method for preventing or treating arteriosclerosis by administering an effective amount of the peptide and an arteriosclerosis therapeutic substance conjugated thereto to a subject in need thereof.

Still another aspect of the present invention is to provide a method for preventing or treating a stroke by administering an effective amount of the peptide and a stroke therapeutic substance conjugated thereto to a subject in need thereof.

### Technical Solution

In accordance with an aspect of the present invention, there is provided a cyclic peptide (cyclo[Cys-Gln-Arg-Pro-Pro-Arg-Cys] peptide) consisting of the amino acid sequence represented by SEQ ID NO: 2 and specifically binding to apoptotic cells.

In accordance with another aspect of the present invention, there are provided a composition containing the peptide as an active ingredient for detecting apoptotic cells and a method for detecting apoptotic cells using the peptide.

In accordance with still another aspect of the present invention, there is provided a composition containing the peptide as an active ingredient for imaging an affected part by an apoptosis-related disease.

In accordance with still another aspect of the present invention, there are provided a composition comprising the peptide as an active ingredient for screening an initial drug response of a test substance having apoptosis-inducing activity in a subject afflicted with an abnormal cell proliferation-related disease; and a method for screening an initial drug response of a test preparation having apoptosis-inducing activity in a subject afflicted with an abnormal cell proliferation-related disease.

In accordance with still another aspect of the present invention, there is provided a composition comprising the peptide as an active ingredient for delivering a drug for an apoptosis-related disease.

In accordance with still further another aspect of the present invention, there is provided a method for treating a tumorous disease, the method comprising administering an effective amount of the peptide and an anti-tumor preparation conjugated thereto to a subject in need thereof.

In accordance with still another aspect of the present invention, there is provided a method for preventing or treating a neurodegenerative disease, the method comprising administering an effective amount of the peptide and a neurodegenerative disease therapeutic substance conjugated thereto to a subject in need thereof.

In accordance with still another aspect of the present invention, there is provided a method for preventing or treating myocardial infarction, the method comprising administering an effective amount of the peptide and a myocardial infarction therapeutic substance conjugated thereto to a subject in need thereof.

In accordance with still another aspect of the present invention, there is provided a method for preventing or treating arteriosclerosis, the method comprising administering an effective amount of the peptide and an arteriosclerosis therapeutic substance conjugated thereto to a subject in need thereof.

In accordance with still another aspect of the present invention, there is provided a method for preventing or treating stroke, the method comprising administering an effective amount of the peptide and a stroke therapeutic substance conjugated thereto to a subject in need thereof.

Hereinafter, the present invention will be described in detail.

As used herein, the term “apoptosis” refers to a phenomenon that causes unnecessary or dangerous cells to die by themselves for the maintenance of individual life. As used herein, the term “apoptotic cells” is meant to encompass all cells which have undergone, progressed, or completed apoptosis, but preferably, may refer to cells in a substantial cell death state due to the completion of the progression of the apoptosis.

The present invention provides a cyclo[Cys-Gln-Arg-Pro-Pro-Arg-Cys] peptide specifically binding to apoptotic cells.

As used herein, the cyclo[Cys-Gln-Arg-Pro-Pro-Arg-Cys] peptide means a cyclic peptide consisting of the amino acid sequence represented by SEQ ID NO: 2, and may be also interchangeably indicated as a cyclic peptide, a cyclic form of ApoPep-1, or the like, in the specification of the present invention. Preferably, the cyclo[Cys-Gln-Arg-Pro-Pro-Arg-Cys] peptide according to the present invention may have a structure of Chemical Formula 1 below.

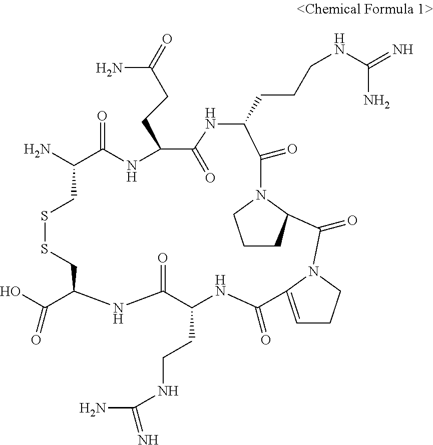 [SMILES: N=C(N)NCCC[C@H]1NC(=O)[C@H](CCC(N)=O)NC(=O)[C@@H](N)CSSC[C@H](C(=O)O)NC(=O)[C@@H](CCNC(=N)N)NC(=O)C2=CCCN2C(=O)[C@H]2CCCN2C1=O]

The peptide of the present invention may be derived from a natural source, and may be synthesized by a known peptide synthesis method. In addition, the peptide of the present invention includes not only a peptide having a natural amino acid sequence, but also an amino acid sequence variant thereof within the scope of the present invention. In the present invention, the amino acid sequence variant of the peptide means a peptide having a different sequence due to the deletion, insertion, or non-conservative or conservative substitution of at least one amino acid residue, or the substitution of an amino acid analog, or a combination thereof derived from the amino acid sequence of SEQ ID NO: 2. The exchange of amino acids, which does not substantially change a molecular activity, is known in the art (H. Neurath, R. L. Hill, The Proteins, Academic Press, New York, 1979).

In some cases, the peptide of the present invention may be modified by phosphorylation, sulfation, acrylation, glycosylation, methylation, farnesylation, or the like.

Korean Registration Patent Nos. 10-0952841 and 10-1077618 by the present inventors disclose that a linear peptide for targeting apoptotic cells, which is represented by SEQ ID NO: 1 and binds to histone H1 present on surfaces of the apoptotic cells, was prepared. However, the linear peptide disclosed in the above patent applications simply showed relatively good detection ability on the basis of the amino acid sequence, but was not meaningful in providing signaling information sensitive enough to image and diagnose an affected part and to understand the relation with actual disease prognosis.

However, the cyclo[Cys-Gln-Arg-Pro-Pro-Arg-Cys] peptide of the present invention has an excellent effect of binding to (or targeting) apoptotic cells as compared with the linear peptide, thereby making it very easy to detect apoptotic cells and image the affected part undergoing apoptosis in vivo, while the detection and imaging signals exhibit very high relevance in view of predicting the prognosis of the disease.

This matter is well shown in Examples below of the present specification.

In one example of the present specification, a cyclo[CQRPPRC] peptide (cyclic form of ApoPep-1) of the present invention was produced by adding Cys (cysteine) residue to the carboxyl terminal of a linear peptide (linear form of ApoPep-1, CQRPPR) represented by SEQ ID NO: 1 to prepare a linear peptide represented by SEQ ID NO: 2 and then performing cyclization through a disulfide bond between the amino terminal and the carboxyl terminal of the linear peptide represented by SEQ ID NO: 2.

In another example of the present invention, the in vitro apoptosis detection effect and the in vitro apoptosis imaging effect of the cyclo[CQRPPRC] peptide (cyclic form of ApoPep-1) of the present invention and the linear form of ApoPep-1 were determined. As a result, the cyclo[CQRPPRC] peptide of the present invention detected apoptosis detected apoptosis more sensitively, compared with annexin V as a control group and the linear form of ApoPep-1 (See Example 1). Especially, the in vivo imaging effect of the cyclo[CQRPPRC] peptide was remarkably improved compared with the linear form of ApoPep-1, while it was found to actually have a strong inverse proportion to tumor prognosis (See Examples 2 to 4).

In addition, according to still another example of the present invention, it was verified that the cyclic form of ApoPep-1 peptide according to the present invention showed the same stability as the linear peptide in serum. Therefore, it was confirmed that the reason that the target efficiency of the cyclic form of ApoPep-1 peptide of the present invention is remarkably high is simply not due to peptide stability (See Example 5). In general, a cyclic structured peptide is known to have improved stability compared with the corresponding linear peptide, and thus, it may be predictable that the cyclic peptide of the present invention may exhibit high intensity of signals due to these characteristics. However, through the serum test, it was suggested that the cyclic peptide of the present invention has a structure of binding better with apoptotic cells (especially, histone H1) than the corresponding linear peptide.

As described above, it was verified that the cyclic peptide of the present invention exhibits an excellent detection effect compared with annexin V which is known as an existing apoptotic probe, while the cyclic peptide of the present invention has an excellent effect of targeting apoptotic cells in tumor cells compared with the corresponding linear peptide, leading to remarkable in vivo imaging and monitoring effects of apoptosis. Therefore, it can be seen that the peptide of the present invention can be used as a composition for detecting apoptotic cells. Further, it can be seen that the peptide of the present invention can be used as a pharmaceutical composition or the like for preventing or treating the disease, which comprises a diagnostic or therapeutic substance for recognizing apoptosis in affected tissues by an apoptosis-related disease, such as a tumor, or a separate substance for treatment of the disease (e.g., tumor).

Therefore, the present invention provides a composition containing the cyclo[Cys-Gln-Arg-Pro-Pro-Arg-Cys] peptide as an active ingredient for detecting apoptotic cells.

In order to determine whether the peptide of the present invention is bound to apoptotic cells and facilitate the detection and quantification of the peptide, the peptide of the present invention may be provided in a labeled state. That is, the peptide of the present invention may be provided by linking (for example, covalently linking or cross-linking) to a detectable label. The detectable label may be a chromogenic enzyme (e.g., peroxidase, alkaline phosphatase), a radioactive isotope (e.g., ⁸¹F, ¹²³I, ¹²⁴I, ¹²⁵I, ³²P, ³⁵S, ⁶⁷Ga), a chromophore, a luminescent material or fluorescer (e.g., FITC, RITC, a fluorescent protein (Green Fluorescent Protein (GFP)); Enhanced Green Fluorescent Protein (EGFP), Red Fluorescent Protein (RFP); Discosoma sp. red fluorescent protein (DsRed); Cyan Fluorescent Protein (CFP), Cyan Green Fluorescent Protein (CGFP), Yellow Fluorescent Protein (YFP), Cy3, Cy5, and Cy7.5), a magnetic resonance imaging material (e.g., gadolinium (Gd), super paramagnetic particles or ultrasuper paramagnetic particles.

Detection methods based on labeling are widely known in the art. For instance, the detection methods may be carried out by the following method. In case a fluorescent material is used as a detectable label, immunofluorescence staining may be employed. In addition, for example, after a sample is reacted with the peptide of the present invention labeled with a fluorescent material, non-bound or non-specifically bound products are removed, and then the florescence by the peptide can be observed under a fluorescent microscope or the intensity of fluorescence may be determined using flow cytometry. In addition, when the detectable label is an enzyme, the absorbance is determined by a color development reaction of a substrate through an enzyme reaction; and when the detectable label is a radioactive material, detection is conducted by measuring the amount of radiation emission. In addition, the detected result may be imaged according to a known imaging method according to the detection label. For example, the cyclic peptide of the present invention may be used as a probe for an imaging (or detecting) means, such as single photon emission computed tomography (SPECT) or PET imaging.

In addition, the present invention provides a method for detecting apoptotic cells, the method comprising: (a) mixing cyclo[Cys-Gln-Arg-Pro-Pro-Arg-Cys] peptides with a sample; (b) removing the peptides that are unbound or non-specifically bound; and (c) determining a binding or non-binding of the peptides and a binding position of the peptides. Here, in step (c), the detection method for a peptide, which is performed in order to investigate whether the cyclo[Cys-Gln-Arg-Pro-Pro-Arg-Cys] peptide of the present invention binds with apoptotic cells, and the binding position of the peptide, may be performed by the method described above or a known method.

As used herein, the term “sample” refers to a biological sample, and encompasses blood and biology-originated other liquid samples, biopsy specimens, solid tissue samples such as tissue culture, or cells derived therefrom. The sample may be obtained from an animal, preferably a mammal. The sample may be pre-treated before the use for detection. For example, the pre-treatment may include extraction, concentration, inactivation of interfering ingredients, addition of reagents, and the like.

The cyclo[Cys-Gln-Arg-Pro-Pro-Arg-Cys] peptide of the present invention specifically binds to apoptotic cells, and thus can image an affected part undergoing apoptosis together with any labeling means (imaging means). Therefore, the present invention provides a composition for imaging an affected part by an apoptosis-related disease, the composition comprising the cyclo[Cys-Gln-Arg-Pro-Pro-Arg-Cys] peptide as an active ingredient.

Here, the imaging and diagnosis of a disease may be used not only for an initial diagnosis of a disease, but also for monitoring a disease progress, a treatment progress, a response to a therapeutic substance, and the like. The peptide of the present invention may be provided in a labeled state in order to investigate the binding or non-binding thereof and facilitate the detection and quantification thereof. This aspect has been described above.

Herein, the term “apoptosis-related disease” refers to a disease encompassing apoptotic activity increased above a normal level, as a major symptomatic feature shown in an affected part. The type of apoptosis-related disease is not limited as long as it is a known apoptosis-related disease, including, for example, a neoplastic disease (cancer), a neurodegenerative disease, stroke, myocardial infarction, arteriosclerosis, retinal disease, and organ transplant rejection.

The neoplastic disease may be brain cancer, neuroendocrine cancer, stomach cancer, lung cancer, breast cancer, ovarian cancer, liver cancer, bronchial cancer, nasopharyngeal cancer, laryngeal cancer, pancreatic cancer, bladder cancer, adrenal gland cancer, colorectal cancer, colon cancer, cervical cancer, prostate cancer, bone cancer, skin cancer, thyroid cancer, parathyroid cancer, and ureteral cancer, but is not limited thereto.

The neurodegenerative disease may be Alzheimer's disease, Parkinson's disease, Huntington's disease, Amyotrophic lateral sclerosis, and Niemann-Pick disease, but is not limited thereto.

In addition, the present invention provides a composition for screening an initial drug response of a test preparation having apoptosis-inducing activity in a subject afflicted with an abnormal cell proliferation-related disease, the composition comprising the cyclo[Cys-Gln-Arg-Pro-Pro-Arg-Cys] peptide as an active ingredient.

As used herein, the term “abnormal cell proliferation-related disease” refers to a disease caused due to abnormal proliferation of cells compared with a normal state. The type of abnormal cell proliferation-related disease is not particularly limited as long as it is a known abnormal cell proliferation-related disease, including, for example, a neoplastic disease, a hyperproliferative vascular disease, and the like. The neoplastic disease is as described above.

As used herein, the term “hyperproliferative vascular disease” refers to a disease or disorder caused by excessive proliferation of cells existing in blood vessels, especially, vascular smooth muscle cells. The hyperproliferative vascular disease includes, for example, arteriosclerosis, atherosclerosis, restenosis and stenosis, vascular malformation, vascular access stenosis associated with hemodialysis, transplant arteriopathy, vasculitis, vascular inflammation, DiGeorge syndrome, hereditary hemorrhagic telangiectasia (HHT), cavernous hemangioma, keloid scar, pyogenic granuloma, blistering disease, Kaposi sarcoma, hyperproliferative vitreous syndrome, retinopathy of prematurity, choroidal neovascularization, macular degeneration, diabetic retinopathy, ocular neovascularization, primary pulmonary hypertension, asthma, nasal polyps, inflammatory bowel and periodontal diseases, seroperitoneum, peritoneal adhesion, contraception, endometriosis, uterine bleeding, ovarian cysts, ovarian hyperstimulation, arthritis, rheumatoid arthritis, chronic rheumatism, synovitis, osteoarthritis, osteomyelitis, osteophyte formation, septicemia, vascular leak syndrome, cancer, infectious diseases, or autoimmune diseases. Preferably, the hyperproliferative vascular disease of the present invention is arteriosclerosis, atherosclerosis, restenosis, or stenosis. Atherosclerosis is a disease in which fatty substances are deposited or fibrosis in the inner layer of the artery and the vascular endothelial cell proliferation occurs, resulting in narrowing or clogging of blood vessels to cause a disorder of the blood flow into peripheral blood vessels. Meanwhile, restenosis is a disease in which the blood vessel passage is narrowed after traumatization of the blood vessel walls, and the main cause of the restenosis is the hyperproliferation of blood vessel muscle cells. It has been known that vascular restenosis occurring after arteriosclerosis progress and stent insertion is caused by the proliferation and migration of vascular smooth muscle cells, the secretion of extracellular matrix, or the like (Circulation, 1997, 95, 1998-2002; J. Clin. Invest. 1997, 99, 2814-2816; Cardiovasc. Res. 2002, 54, 499-502). Therefore, researches have been extensively made on a drug inhibiting the proliferation of vascular smooth muscle cells to prevent artheriosclerosis development and vascular restenosis (J. Am. Coll. Cardiol., 2002, 39, 183-193).

As used herein, the term “test preparation” includes any substance, molecule, element, compound, entity, or a combination thereof. For example, the term encompasses, but is not limited to, a protein, a polypeptide, a small organic molecule, a polysaccharide, a polynucleotide, and the like. Moreover, the term may be a natural product, a synthetic compound, a chemical compound, or a combination of two or more materials. Unless otherwise specified, the terms “preparation”, “material”, and “compound” can be used interchangeably.

As used herein, the term “test preparation having apoptosis-inducing activity” refers to a material that induces apoptosis of abnormally proliferating cells in an affected part of a subject having the abnormal cell proliferation-related diseases, ultimately exhibiting therapeutic activity, and most preferably, a preparation that does not show a substantial apoptosis-inducing action on normal cells and substantially exhibits an apoptosis-inducing action only on the abnormally proliferating cells in the affected part may be preferable. With respect to the test preparation having apoptosis-inducing activity, a person skilled in the art can selectively use the type of drug according to the disease. For example, when the abnormal cell proliferation-related disease is a neoplastic disease (particularly cancer), the test preparation may be a known anti-tumor agent (anti-cancer agent).

As used herein, the term “drug response” refers to a state change of improvement in the symptom of an affected part by a drug in a subject suffering from a particular disease. In the present invention, the term, preferably, means a state in which apoptosis is increased by a drug in an affected part. The term “response” may be understood as susceptibility or sensitivity, while those terms can be used interchangeably. Therefore, the meaning of a test preparation having a drug response (or susceptibility) is that the likelihood of its therapeutic efficacy is higher compared with that of other test preparation having no drug response (susceptibility). Specifically, for example, when the particular disease is a tumor (cancer), the term “drug response” is understood as a tumor response to a drug. The tumor response means that some patients show therapeutic effects, whereas other patients show no therapeutic effects, even though the clinical histopathological characteristics are the same. There exist clinically significant biological differences among tumors which we do not currently understand and can only be seen after treatment.

As used herein, the term “screening a drug response” refers to selecting a test preparation which shows a response of symptom improvement in an affected part by a particular disease.

As used herein, the term “initial” refers to a typical early stage in the administration of a test preparation in view of a dictionary definition. Since the general dose and period of administration vary depending on the types of disease and test preparation (drug), the specific date or time in a typical early stage in the administration of the test preparation may be varied, while a person skilled in the art is able to appropriately modify the date or time according to the condition of a subject to be administered. For example, the “initial” may be 1 to 30 days from the beginning of administration of a test preparation, preferably 1 to 15 days, and most preferably, 1 to 7 days from the beginning of administration of a test preparation.

Generally, there is an individual difference in view of the drug response (susceptibility) to a certain disease, and such an individual difference varies depending on the genotype of the individual and the type of disease. In many aspects, such as treatment efficiency, time, and cost burden, it is very important to early predict the response and effectiveness of a particular treatment method or drug in determining the patient's treatment strategy. In addition, such an early prediction and determination of the drug response in an abnormal cell proliferation-related disease, particularly cancer (tumor), is important since it is closely related to a determination on whether the cancer (tumor) acquires resistance to an anticancer drug as well as therapeutic effects by a drug. With respect to a detection means, proteins and peptides targeting various substances are used in the art, but in practice, in order for the peptides to be meaningfully used to give an imaging means for an affected part and the diagnostic information of disease symptoms, a significant correlation is required between the measured signal information and the prognosis of actual disease symptoms.

Therefore, many peptides are merely used for simple detection purposes, while there is a limitation in actually using the peptides for statistically predicting the prognosis of a disease on the basis of detection or imaging results. However, the use of the cyclic peptide of the present invention has an effect of actually predicting the prognosis of disease symptoms by a therapeutic substance (test preparation) used for treatment since there is a significant correlation between a signal (fluorescence signal) obtained through body images using the peptide of the present invention in the response of an affected part at an early stage of drug treatment and a prognosis of the actual relief of symptoms of an affected part after completion of the drug treatment.

This is well described in Examples of the invention.

In an Example of the present invention, at 1 week and 2 weeks after the initiation of anti-cancer treatment on mice, a fluorescence-labeled cyclic form of ApoPep-1 (cyclo[Cys-Gln-Arg-Pro-Pro-Arg-Cys] peptide of the present invention) and linear form of ApoPep-1 from which the cyclic peptide is originated were each injected through the tail vein to obtain an in vivo imaging fluorescence signals, and then the correlation between the obtained fluorescence signal and the prognosis of later-on tumor volume reduction (after 3 weeks) was examined through linear regression analysis. As a result, it was verified that the fluorescence signal obtained by NIR fluorescence imaging using the cyclic form of ApoPep-1 of the present invention showed a very high correlation inversely with the prognosis of later-on tumor volume reduction in one week after the initiation of the anti-cancer treatment on the mice.

Therefore, the cyclic form of ApoPep-1 peptide of the present invention is excellent in early prediction of the therapeutic prognosis of actual symptoms by a therapeutic substance (test preparation).

Therefore, the present invention provides a method for screening an initial drug response of a test preparation in a subject afflicted with an abnormal cell proliferation-related disease, the method comprising:

(a) treating a target tissue of an affected part isolated from a subject with a test preparation having apoptosis-inducing activity, wherein the subject is afflicted with an abnormal cell proliferation-related disease;

(b) treating the test preparation-treated target tissue of step (a) and a control target tissue treated without a test preparation, with cyclo[Cys-Gln-Arg-Pro-Pro-Arg-Cys] peptide labeled with a labeling means; and

(c) detecting and comparing the labeling means in the peptide-treated target tissues in step (b).

The “method for screening an initial drug response of a test preparation in a subject having an abnormal cell proliferation-related disease, the method comprising steps (a) to (c)” may be performed by further comprising (d) determining the target tissue as being responsive to the test preparation if an increased level of the labeling means is detected in the test preparation-treated target tissue in comparison with the control target tissue.

In addition, in the above method, the labeling means and the detection method therefor are as described above, and may be implemented by a known method.

As used herein, the term “affected part” refers to a site in which a disease or a wound occurs.

In addition, the peptide of the present invention has an excellent effect of specifically binding to apoptotic cells, and thus can be used as an intelligent drug delivery system for selectively delivering a drug to the apoptotic cells (ultimately to the diseased site where the apoptotic cells are present). Accordingly, provided is a composition comprising the peptide of the present invention as an active ingredient for delivering a drug for an apoptosis-related disease.

Therefore, the composition for delivering a drug according to the present invention, may be specific to an apoptosis-related disease, such as a neoplastic disease, a neurodegenerative disease, stroke, myocardial infarction, or arteriosclerosis. The apoptosis-related disease is as described above.

When the cyclic peptide of the present invention contained in the composition for drug delivery according to the present invention is used for treatment in linkage with medicines, such as an anti-tumor substance, a neurodegenerative disease therapeutic substance, a stroke therapeutic substance, a myocardial infarction therapeutic substance, an arteriosclerosis therapeutic substance, and the like, the preparations are selectively delivered to only disease sites (affected parts), such as tumor cells, neurodegenerative disease sites, stroke sites, myocardial infarction sites, arteriosclerosis sites, and the like, thereby increasing the efficacy of those substances while significantly reducing adverse side effects on normal tissues.

Therefore, the present invention provides: a pharmaceutical composition for preventing and treating a neoplastic disease, the pharmaceutical composition comprising, as an active ingredient, the peptide of the present invention and an anti-tumor substance conjugated thereto; a composition for preventing and treating a neurodegenerative disease, the composition comprising, as an active ingredient, the peptide of the present invention and a neurodegenerative disease therapeutic substance conjugated thereto; a pharmaceutical composition for preventing and treating myocardial infarction, the pharmaceutical composition comprising, as an active ingredient, the peptide of the present invention and a myocardial infarction therapeutic substance conjugated thereto; a pharmaceutical composition for preventing and treating arteriosclerosis, the pharmaceutical composition comprising, as an active ingredient, the peptide of the present invention and an arteriosclerosis therapeutic substance conjugated thereto; and a pharmaceutical composition for preventing and treating stroke, the pharmaceutical composition comprising, as an active ingredient, the pharmaceutical composition comprising the peptide of the present invention and a stoke therapeutic substance conjugated thereto.

The type of anti-tumor substance that may be linked with the peptide of the present invention is not particularly limited as long as it is a known tumor therapeutic material. For example, the anti-tumor substance may be at least one selected from the group consisting of paclitaxel, doxorubicin, vincristine, daunorubicin, vinblastine, actinomycin-D, docetaxel, etoposide, teniposide, bisantrene, homoharringtonine, Gleevec (STI-571), cisplain, 5-fluouracil, adriamycin, methotrexate, busulfan, chlorambucil, cyclophosphamide, melphalan, nitrogen mustard, nitrosourea, streptokinase, urokinase, alteplase, angiotensin II inhibitor, aldosterone receptor inhibitor, erythropoietin, NMDA (N-methyl-d-aspartate) receptor inhibitor, lovastatin, rapamycin, Celebrex, Ticlopin marimastat, and Trocade.

In addition, a neurodegenerative disease therapeutic substance, a stroke therapeutic substance, a myocardial infarction therapeutic substance, and an arteriosclerosis therapeutic substance can be used without limitation as long as these are used in the treatment of corresponding diseases. For example, the neurodegenerative disease therapeutic substance that can be linked with the peptide of the present invention may be, as a brain nerve cell protector, an N-methyl-d-aspartate (NMDA) receptor inhibitor, an acetylcholine esterase inhibitor, an anti-amyloid protein, or the like, and examples thereof may be donepezil, galantamine, tacrine, memantine, or the like. In addition, there are drugs, such as streptokinase, urokinase, and alteplase, as thrombolytic drugs that are used to remove blood clots blocking blood vessels in stroke and myocardial infarction diseases. In addition, there are an angiotensin II inhibitor, an aldosterone receptor inhibitor, and erythropoietin, as myocardial cell protective agents. In addition, there are: Lovastatin as a drug inhibiting cholesterol synthesis and lowering the blood cholesterol level; Rapamycin as a drug reducing the proliferation of vascular smooth muscle cells; Celebrex as an anti-inflammatory drug; ticlopine as a platelet aggregation inhibitor; and Marimastat and Trocade as matrix metalloprotease inhibitors. The linking of such a substance and the peptide of the present invention may be carried out through a method known in the art, for example, covalent linking, cross-linking, or the like. To this end, the cyclic peptide of the present invention may be, if necessary, chemically modified within the scope in which its activity is not lost. The amount of the cyclic peptide of the present invention contained in the composition of the present invention may vary depending on the type and amount of the therapeutic agent to be linked.

In the pharmaceutical composition of the present invention, the cyclic peptide of the present invention may be provided in a labeled state to facilitate the investigation whether the cyclic peptide is bound to a target organ, the detection thereof, and the quantification thereof. The above description may be referred to in this regard.

Meanwhile, the pharmaceutical composition according to the present invention may be provided by being formulated in a pure form of the peptide or a suitable form together with a pharmaceutically acceptable carrier. The term “pharmaceutically acceptable” composition refers to a non-toxic composition that is physiologically acceptable and does not cause an allergic response, such as gastrointestinal disorder or vertigo, or similar responses, when administered to humans. The carrier includes all types of a solvent, a dispersion medium, an oil-in-water or water-in-oil emulsion, an aqueous composition, liposomes, microbeads microsomes, biodegradable nanoparticles

Meanwhile, the pharmaceutical composition of the present invention may be formulated with an appropriate carrier according to the route of administration. The route of administration of the pharmaceutical composition according to the present invention may be orally or parenterally, but is not limited thereto. Examples of the route of parenteral administration include several routes, such as transdermal, intranasal, intraperitoneal, intramuscular, subcutaneous, and intravenous routes.

The pharmaceutical composition of the present invention, when orally administered, may be formulated, together with a suitable carrier for oral administration, in the form of a powder, granules, a tablet, a pill, a sugar coated tablet, a capsule, a liquid, a gel, a syrup, a suspension, a wafer, or the like by a method known in the art. Examples of the suitable carrier may include: sugars including lactose, dextrose, sucrose, sorbitol, mannitol, xylitol, erythritol, and maltitol; starches including corn starch, wheat starch, rice starch, and potato starch; celluloses including cellulose, methyl cellulose, sodium carboxy methyl cellulose, and hydroxypropyl methyl cellulose; and a filler, such as gelatin or polyvinyl pyrrolidone. In some cases, cross-linked polyvinyl pyrrolidone, agar, alginic acid, or sodium alginate may be added as a disintegrant. Further, the pharmaceutical composition of the present invention may further contain an anti-coagulant, a slipping agent, a wetting agent, a favoring agent, an emulsifier, and a preservative.

As for the parenteral administration, the pharmaceutical composition of the present invention may be formulated in a dosage form of an injection, a transdermal administration preparation, and a nasal inhalant, together with a suitable parenteral carrier, by a method known in the art. The injection needs to be essentially sterilized, and be protected from the contamination of microorganisms, such as bacteria and fungi. Examples of the suitable carrier for the injection may include, but are not limited to, water, ethanol, polyols (e.g., glycerol, propylene glycol, liquid polyethylene glycol, etc.), mixtures thereof, and/or solvents or dispersive media containing vegetable oils. More preferably, Hanks' solution, Ringer's solution, phosphate buffered saline (PBS) or sterile water for injection containing triethanolamine, or an isotonic solution (such as 10% ethanol, 40% propylene glycol, or 5% dextrose) may be used as a suitable carrier. In order to protect the injection from microbial contamination, the injection may further contain various antibiotics and antifungal reagents, such as paraben, chlorobutanol, phenol sorbic acid, and thimerosal. In most cases, the injection may further contain an isotonic agent, such as sugar or sodium chloride. These preparations are described in the document, which is a formulary generally known in pharmaceutical chemistry (Remington's Pharmaceutical Science, 15th Edition, 1975, Mack Publishing Company, Easton, Pa.).

In the case of a nasal administration preparation, the compound used according to the invention may be conveniently delivered in the form of aerosol spray from a pressurized pack or a nebulizer, using a suitable propellant, for example, dichlorofluoromethane, trichlorofluoromethane, dichlorotetrafluoroethane, carbon dioxide, or other suitable gases. In the case of a pressurized aerosol, the dosing unit may be determined by providing a valve that delivers a measured quantity. For example, a gelatin capsule and a cartridge used in an inhaler or an insufflator may be formulated to contain a compound, and a powder mixture of proper powder materials, such as lactose or starch.

The following document may be referred to for other examples of the pharmaceutically acceptable carrier (Remington's Pharmaceutical Sciences, 19th ed., Mack Publishing Company, Easton, Pa., 1995).

In addition, the pharmaceutical composition according to the present invention may further contain at least one buffer (for example, saline solution or PBS), a carbohydrate (for example, glucose, mannose, sucrose, or dextran), a stabilizer (for example, sodium bisulfate, sodium sulfite, or ascorbic acid), an antioxidant, a bacteriostatic agent, a chelating agent (for example, EDTA or glutathione), an adjuvant (for example, aluminum hydroxide), a suspension agent, a thickener, and/or a preservative (for example, benzalkonium chloride, methyl- or propyl-paraben, and chlorobutanol).

In addition, the pharmaceutical composition of the present invention may be formulated by a method known in the art to provide rapid, continuous, or delayed release of an active ingredient after the pharmaceutical composition is administered to a mammal.

The pharmaceutical composition formulated by the method above may be administered at an effective amount through several routes including oral, transdermal, subcutaneous, intravenous, and intramuscular routes. As used herein, the term “effective amount” refers to an amount of a compound or an extract which makes it possible to trace a diagnostic or therapeutic effect when the pharmaceutical composition is administered to a patient. The dose of the pharmaceutical composition according to the present invention may be appropriately selected depending on the route of administration, the subject of administration, the subject disease and severity thereof, age, gender, weight, individual differences, and disease conditions. Preferably, the content of an active ingredient in the pharmaceutical composition containing the peptide of the present invention may be varied according to the extent of a disease, but an effective dose of 1-1000 mg on the basis of an adult may be repeatedly administered several times a day.

In addition, the present invention provides a method for treating a neoplastic disease, the method comprising administering an effective amount of the cyclic peptide of the present invention and an anti-tumor substance conjugated thereto to a subject in need thereof.

The present invention provides a method for preventing or treating a neurodegenerative disease, the method comprising administering an effective amount of the cyclic peptide of the present invention and a neurodegenerative disease therapeutic substance conjugated thereto to a subject in need thereof.

The present invention provides a method for preventing or treating myocardial infarction, the method comprising administering an effective amount of the cyclic peptide of the present invention and a myocardial infarction therapeutic substance conjugated thereto to a subject in need thereof.

The present invention provides a method for preventing or treating arteriosclerosis, the method comprising administering an effective amount of the cyclic peptide of the present invention and an arteriosclerosis therapeutic substance conjugated thereto to a subject in need thereof.

The present invention provides a method for preventing or treating stroke, the method comprising administering an effective amount of the cyclic peptide of the present invention and a stroke therapeutic substance conjugated thereto to a subject in need thereof

The neoplastic disease, neurodegenerative disease, myocardial infarction, arteriosclerosis, and stroke pertain to apoptosis-related disease, and these are as described above.

The anti-tumor substance, neurodegenerative disease therapeutic substance, myocardial infarction therapeutic substance, arteriosclerosis therapeutic substance, and stroke therapeutic substance may be used without limitation as long as the therapeutic substances are used in the treatment of these diseases, while those therapeutic substances are as described above.

As used herein, the term “effective amount” of the substance or preparation refers to its amount showing effects of treating and preventing said disease, respectively, upon being administered to a patient. As used herein, the term “subject” refers to an animal, preferably a mammal, in particular an animal including a human being, while the subject may be cells, tissue, and organ, or the like originated therefrom. The subject may be a patient in need of treatment.

### Advantageous Effects

However, the cyclic peptide (cyclo[Cys-Gln-Arg-Pro-Pro-Arg-Cys] peptide) of the present invention including the amino acid sequence represented by SEQ ID NO: 2 has an excellent effect of binding to (or targeting) apoptotic cells as compared with the corresponding linear peptide, thereby facilitating the detection of apoptotic cells and the in vivo imaging of the affected part undergoing apoptosis, while the detection and imaging signals exhibit a very high relevance in view of predicting the prognosis of a disease. Therefore, the cyclic peptide of the present invention can diagnose a response of a therapeutic drug to an abnormal cell proliferation-related disease at an early stage by binding with an imaging material, and can be used for the purpose of selectively delivering a drug to an apoptosis-relating disease tissue by binding with a therapeutic material.

### BRIEF DESCRIPTION OF THE DRAWINGS

FIG. 1 quantitatively shows in vitro detection results of apoptotic cells using linear form of ApoPep-1(A), cyclic form of ApoPep-1(B), and Annexin V(C) after treatment of cells with either cisplatin or cetuximab alone, or in combination thereof (PBS: control group treated without anti-cancer drug, CPT: group treated with cisplatin alone, CET: group treated with cetuximab alone, CPT+CET: group treated with cisplatin and cetuximab in combination).

FIG. 2 shows fluorescence intensity when mice were treated with either cisplatin or cetuximab alone, or in combination thereof at the 1st and 2nd rounds and then apoptotic cells were subjected to in vivo NIR fluorescence imaging using linear form of ApoPep-1 (A) and cyclic form of ApoPep-1 (B), respectively (PBS: group treated without anti-cancer drug, CPT: group treated with cisplatin alone, CET: group treated with cetuximab alone, CPT+CET: group treated with cisplatin and cetuximab in combination, 1st: group treated with the anti-cancer drug at the 1st round (1 week), 2nd: group treated with the anti-cancer drug at the 2nd round (2 week)).

FIG. 3 shows representative images when mice were treated with either cisplatin or cetuximab alone, or in combination thereof at the 1st and 2nd rounds and then apoptotic cells were subjected to in vivo NIR fluorescence imaging using linear form of ApoPep-1 (C) and cyclic form of ApoPep-1 (D), respectively (PBS: group treated without anti-cancer drug, CPT: group treated with cisplatin alone, CET: group treated with cetuximab alone, CPT+CET: group treated with cisplatin and cetuximab in combination, 1st: group treated with the anti-cancer drug at the 1st round (1 week), 2nd: group treated with the anti-cancer drug at the 2nd round (2 week)).

FIG. 4 shows changes in tumor volume up to 3 weeks after anti-cancer treatment of mice, which were treated with either cisplatin or cetuximab alone, or in combination thereof at the 1st and 2nd rounds and apoptosis was detected at an early stage using linear form of ApoPep-1 (A) and cyclic form of ApoPep-1 (B), respectively (PBS: group treated without anti-cancer drug, CPT: group treated with cisplatin alone, CET: group treated with cetuximab alone, CPT+CET: group treated with cisplatin and cetuximab in combination, Arrows indicate time points for anti-cancer treatment).

FIG. 5 shows a tumor weight measured by taking a tumor from mice 3 weeks after anti-cancer treatment when the mice were treated with either cisplatin or cetuximab alone, or in combination thereof at the 1st and 2nd rounds and apoptosis was detected at an early stage using linear form of ApoPep-1 (C) and cyclic form of ApoPep-1 (D), respectively (PBS: group treated without anti-cancer drug, CPT: group treated with cisplatin alone, CET: group treated with cetuximab alone, CPT+CET: group treated with cisplatin and cetuximab in combination, ▪, ▴, ▾, ♦ indicate measured values for each subject of a subgroup (n=3) and - indicates a mean value).

FIG. 6 shows TUNEL staining results of tumor tissues taken from mice 3 weeks after anti-cancer treatment when the mice were treated with either cisplatin or cetuximab alone, or in combination thereof at the 1st and 2nd rounds and apoptosis was detected at an early stage using linear form of ApoPep-1 (E) and cyclic form of ApoPep-1 (F), respectively (Green: apoptotic cells; Blue: nucleus, PBS: group treated without anticancer drug, CPT: group treated with cisplatin alone, CET: group treated with cetuximab alone, CPT+CET: group treated with cisplatin and cetuximab in combination, scale bar at the bottom of each image indicates 50 μm).

Panels A and C of FIG. 7 show linear regression analysis results of the correlation between the in vivo fluorescence intensity and the prognosis of tumor volume when the mice were treated with anticancer drug (either cisplatin or cetuximab alone, or in combination thereof) at the 1st round and subjected to NIR fluorescence imaging using linear form of ApoPep-1 (A) and cyclic form of ApoPep-1 (C), respectively.

Panels B and D of FIG. 7 show linear regression analysis results of the correlation between the in vivo fluorescence intensity and the prognosis of tumor volume when the mice were treated with anti-cancer drugs (either cisplatin or cetuximab alone, or in combination thereof) at the 2nd round and subjected to NIR fluorescence imaging using linear form of ApoPep-1 (B) and cyclic form of ApoPep-1 (D), respectively.

FIG. 8 shows C18 reverse-phase FPLC analysis results in the order of time of peptides collected after linear form of ApoPep-1 (A) and cyclic form of ApoPep-1 (B) were incubated in mouse serum for 0-24 hours (Y axis indicates the absorbance unit at 215 nm and X axis indicates the retention time. Arrows indicate the peak of linear or cyclic form of ApoPep-1 peptide).

FIG. 9A shows MS spectra for peptide peak fractions obtained by incubating linear form of ApoPep-1 in mouse serum for 24 hours, collecting the peptides, and performing C18 reverse-phase FPLC. FIG. 9B shows MS results for the linear form of ApoPep-1 peptide in an initial synthetic state not incubated in serum (Arrows indicate the peak of the linear form of ApoPep-1 peptide). It can be seen through the comparision of FIG. 9A and FIG. 9B that linear form of ApoPep-1 was stably present in serum even after incubation for 24 hours.

FIG. 9C shows MS spectra for peptide peak fractions obtained by incubating cyclic form of ApoPep-1 in mouse serum for 24 hours, recovering the peptides, and performing C18 reverse-phase FPLC. FIG. 9D shows MS results for the cyclic form of ApoPep-1 peptide in an initial synthetic state not incubated in serum (Arrows indicate the peak of the cyclic form of ApoPep-1 peptide). It can be seen through the comparision of FIG. 9C and FIG. 9D that cyclic form of ApoPep-1 was stably present in serum even after incubation for 24 hours.

### MODE FOR CARRYING OUT THE INVENTION

Hereinafter, the present invention will be described in detail.

However, the following examples are merely for illustrating the present invention and are not intended to limit the scope of the present invention.

<Materials and Methods>

1. Synthesis and Fluorescence Labeling of Peptides

Linear form of ApoPep-1 (CQRPPR, SEQ ID NO: 1) and cyclic form of ApoPep-1 (cyclo[CQRPPRC] of the present invention, SEQ ID NO: 2, cyclization via disulfide bonding between amino and carboxy termini) peptides were synthesized by Peptron Inc. (Daejeon, Korea.), and were purified to >95% purity using high performance liquid chromatography (HPLC). Peptides were labeled with FITC (fluorescein isothiocyanate) or FPR675 near-infrared (NIR) fluorescence dye (Bioacts, Inc., Incheon, Korea.).

2. In Vitro Binding of Peptides to Apoptotic Cells

SNU16 human stomach cancer cell line was purchased from KCLB (Seoul, Korea). To induce apoptosis, cells were treated with cisplatin (300 ng/ml), cetuximab (200 ug/ml), or cisplatin (300 ng/ml) plus cetuximab (200 mg/ml) in combination for 24 hours. The concentrations of cisplatin and cetuximab were chosen according to the previous reports (Choi C H, Cha Y J, An C S, Kim K J, Kim K C, et al. (2004) Molecular mechanisms of heptaplatin effective against cisplatin-resistant cancer cell lines: less involvement of metallothionein. Cancer Cell Int 4: 6.; Yun J, Song S H, Park J, Kim H P, Yoon Y K, et al. (2012) Gene silencing of EREG mediated by DNA methylation and histone modification in human gastric cancers. Lab Invest 92: 1033-1044.). After treatment, cells were incubated with fluorescein isothiocyanate (FITC)-conjugated linear or cyclic form of ApoPep-1 peptide at 4□ for 1 hour. As control, cells were stained with Alexa488-conjugated annexin V (Life technologies, Carlsbad, Calif.) for 15 min at room temperature. Percentages of fluorescent (the liner or cyclic form of ApoPep-1 peptide-bound or annexin V-bound fluorescence) cells were calculated by measurement and analysis methods through the selection of the fluorescent signals (the liner or cyclic form of ApoPep-1 peptide-bound or annexin V-bound fluorescence), which were shown in the respective cells when cells in an emulsion state pass through a constant fluorescence detection zone, using flow cytometry (Fluorescence-activated cell sorting (FACS), FACS calibur, BD Biosciences, MA, USA).

3. Anti-Tumor Treatment of Mice and Tumor Size Measurement

All animal experiments were performed in compliance with institutional guidelines and according to the animal protocol approved by the guideline of the Institutional Animal Care and Use Committee (IACUC) of Kyungpook National University (permission No. KNU 2012-15).

Eight-week old female athymic (nu/nu) Balb/c mice were purchased from Orient laboratories (Seongnam, Korea) and were housed under specific-pathogen-free (SPF) conditions with laboratory chow and water ad libitum. Stomach tumor xenografts were established by subcutaneously injecting 1×10⁷ SNU-16 cells in 100 ml saline into the right flank. Tumors were allowed to reach 50-60 mm³ of volume before randomization and initiation of treatment. Treatment of tumor-bearing mice with cisplatin and cetuximab was conducted according to a previously described protocol (Steiner P, Joynes C, Bassi R, Wang S, Tonra J R, et al. (2007) Tumor growth inhibition with cetuximab and chemotherapy in non-small cell lung cancer xenografts expressing wild-type and mutated epidermal growth factor receptor. Clin Cancer Res 13: 1540-1551.). Mice were divided into four treatment groups (n=6 per group) and treated for two weeks: 1) control treated with phosphate buffered saline (PBS, control); 2) cisplatin treatment group (5 mg/kg, intraperitoneal (i.p.) injection, once per week for total two injections); 3) cetuximab treatment group (1.5 mg/kg, i.p., twice per week for total four injections); 4) cisplatin treatment group (5 mg/kg, i.p., once per week for total two injections) plus cetuximab (1.5 mg/kg, i.p., twice per week for total four injections). One round of anticancer drug treatment was conducted in a manner of injection of cisplatin at day 1 per week (the first day of the week on a weekly basis) and cetuximab at day 1 and day 4 per week (the first day and the fourth day of the week on a weekly basis). In the present experiment, a total of two rounds of anticancer drug treatment were conducted for two weeks. Changes in tumor size were measured using an automatic caliper over three weeks. Tumor volumes were calculated using the formula: volume=(length×width×height)/2. Tumor weights were measured after isolation of tumor mass.

4. In Vivo NIR Fluorescence Imaging of Tumor Apoptosis

In vivo NIR fluorescence imaging was performed after the first and second round of treatment. Each treatment group (n=6) was divided into two subgroups (n=3) for imaging with linear and cyclic forms of ApoPep-1, respectively. Linear and cyclic forms of FPR675-labeled ApoPep-1 (1.45 mg/kg and 1.54 mg/kg, respectively; equivalent to 800 nmol/kg for each peptide) was injected through the tail vein into mice. At 90 minutes after injection of the fluorescence-labeled linear and cyclic forms of ApoPep-1 peptide, mice were anesthetized and subjected to imaging. NIR fluorescence (typically, between 650 and 1100 nm) is favored for in vivo optical imaging because of its low tissue absorption and deep tissue penetration properties (Konig K (2000) Multiphoton microscopy in life sciences. J Microsc 200: 83-104). The excitation/emission wavelength of the FPR675 dye used in this study was 675/698 nm. Images were taken using the eXplore Optix optical imaging system (ART Inc., Montreal, Canada), and the acquisition time for a whole-body scanning was 15 minutes per mouse. Fluorescence intensity at region of interest (ROI) was measured using a analysis software provided by the manufacturer (ART Inc.).

5. Histologic Analysis of Apoptosis

After in vivo imaging, mice were euthanized three weeks after the initiation of anticancer drug treatment, and the tumors were removed and frozen quickly in O.C.T. embedding medium (Sakura Finetechnical, Tokyo, Japan). Tissues were cut into 6 um sections, and stained with DAPI (4′,6-diamidino-2-phenylindole) for nucleus counterstaining. Terminal deoxy-nucleotidyl transferase-mediated dUTP nick-end labeling (TUNEL) staining was conducted using the Apoptag Red In Situ Apoptosis Detection kit according to guidelines of the manufacturer (Millipore, Billerica, Mass.). The stained tissue sections were observed under a fluorescence microscope (Carl Zeiss, Jena, Germany).

6. Correlation Analysis Between Fluorescence Intensity and Tumor Volume

At three weeks after the initiation of anticancer drug treatment (endpoint of experiments), tumor volumes were measured and tumors were isolated from the mice for the weight measurement. The correlation between NIR fluorescence intensity and tumor volume was evaluated by the linear regression analysis using the Graphpad software.

7. Stability of Peptides in the Serum

Peptide stability in the serum was examined by the same method referring to the following documents Yoo S A, Bae D G, Ryoo J W, Kim H R, Park G S, et al. (2005) Arginine-rich antivascular endothelial growth factor (anti-VEGF) hexapeptide inhibits collageninduced arthritis and VEGF-stimulated productions of TNF-alpha and IL-6 by human monocytes. J Immunol 174: 5846-5855. Blood from mice was collected, and then serum was collected by centrifugation at 4□, followed by filtration through 0.22 um-pore filter. Linear and cyclic forms of ApoPep-1 peptides (100 ug of peptide contained in 50 uL of PBS) was incubated with 50 ml of filtered serum at 37□ for 24 hours at time intervals (each sample was composed of serum 50 μL+peptide 50 uL (that is, 100 ug)=100 uL, and reacted in each tube according the time of 0 h, 1 h, 4 h, 8 h, 16 h, 24 h). The samples were diluted to 100-fold, and injected in a volume of 100 μl. The flow rate was 0.3 ml/min, and analyzed by C18 reverse phase FPLC using a linear gradient of 20% from 0 to 100% using acetonitrile through a linear graduation of 20% (Vydac protein and peptide C18, 0.1% trifluoroacetate in water for equilibration, and 0.1% trifluoroacetate in acetonitrile for elution) (Life Technologies, Carlsbad, Calif.). The fraction samples collected according to each peak show as a result of C18 reverse phase FPLC were collected, and frozen dried. To identify the identity of the peptide from the profiles of C18 reverse phase FPLC, each peak was collected, and subjected to mass spectrometry (MS) using an MALDI-TOF mass spectrometer (Life Technologies, Carlsbad, Calif.).

8. Statistical Analysis

The statistical significance of differences between experimental and control groups was analyzed using one-way analysis of variance (ANOVA) (* p<0.05, ** p<0.01, ***p<0.001, statistical significance was shown for each drawing).

### Example 1

In Vitro Detection of Apoptosis of Stomach Tumor Cells Using ApoPep-1 after Treatment with Cisplatin and Cetuximab

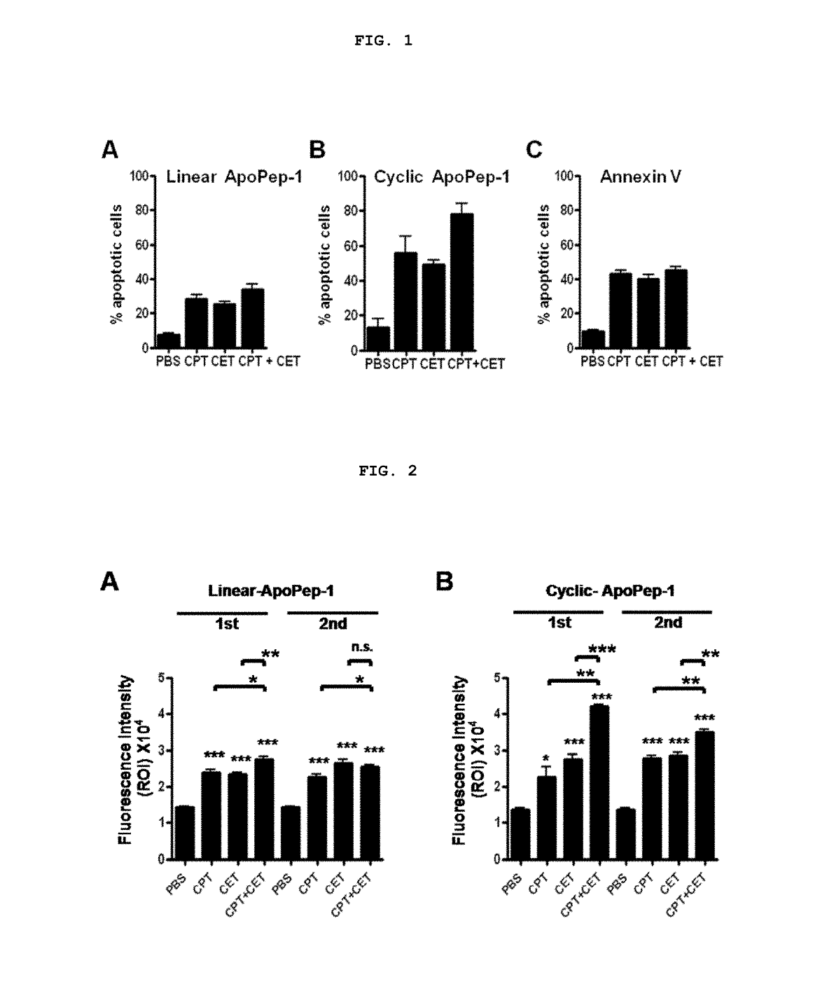

In order to examine the detection of apoptosis according to the structure features of ApoPep-1, stomach tumor cells were treated with cisplatin or cetuximab alone, or cisplatin plus cetuximab in combination, and then incubated with FITC-conjugated linear and cyclic forms of ApoPep-1. Cyclic form of ApoPep-1 (cyclo[CQRPPRC] peptide of the present invention) was prepared by adding cysteine residue at the carboxy terminal of linear form of ApoPep-1 (CQRPPR) and performing cyclization through disulfide bonding. The percentages of apoptotic cells detected by the linear form of ApoPep-1 were approximately 28%, 25%, and 34% in the groups treated with cisplatin alone, cetuximab alone, and cisplatin and cetuximab in combination, respectively (panel A of FIG. 1). The percentages of apoptotic cells detected by the cyclic form of ApoPep-1 were approximately 56%, 49%, and 78% in the groups treated with cisplatin alone, cetuximab alone, and cisplatin and cetuximab in combination, respectively (panel B of FIG. 1). The percentages of apoptotic cells detected by annexin V were approximately 43%, 40%, and 45% in the groups treated with cisplatin alone, cetuximab alone, and cisplatin and cetuximab in combination, respectively (panel C of FIG. 1). These results show that the combined treatment of cisplatin and cetuximab induces apoptosis of stomach tumor cells at higher levels than the treatment of cisplatin or cetuximab alone. Also, these results suggest that the cyclic form of ApoPep-1 more sensitively detects apoptosis of stomach tumor cells than the linear form of ApoPep-1 or annexin V.

### Example 2

In Vivo Imaging of Apoptosis of Stomach Tumor Using ApoPep-1 after Treatment with Cisplatin and Cetuximab

In order to examine in vivo detection and imaging of apoptosis of apoptosis according to the structural feature of ApoPep-1, the fluorescence intensity at tumor by the accumulation of NIR fluorescence dye (FPR675) labeled-ApoPep-1 to tumor tissue was measured after the first and second round of treatment (equivalent to one week and two weeks after the initiation of treatment, respectively). Quantification of fluorescence intensity at tumor site by linear or cyclic form of ApoPep-1 showed that the intensities were significantly higher in groups treated with cisplatin alone, cetuximab alone, and cisplatin and cetuximab in combination, compared with the control group treated without a drug, after the first or second round of treatment (panel A and panel B of FIG. 2). Fluorescence intensities by linear form of ApoPep-1 were higher in the group treated with cisplatin and cetuximab in combination compared with the group treated with cisplatin alone (p<0.05 and p<0.05 after the first and second round of treatment, respectively, panel A of FIG. 2) or cetuximab alone (p<0.01 after the first round of treatment, not significant after the second round of treatment, respectively, panel A of FIG. 2).

Particularly, fluorescence intensities by cyclic form of ApoPep-1 were higher in the group treated with cisplatin and cetuximab in combination compared with the group treated with cisplatin alone (p<0.01 and p<0.01 after the first and second round of drug treatment, respectively, panel B of FIG. 2) or cetuximab alone (p<0.001 and p<0.01 after the first and second rounds of treatment, respectively, panel B of FIG. 2).

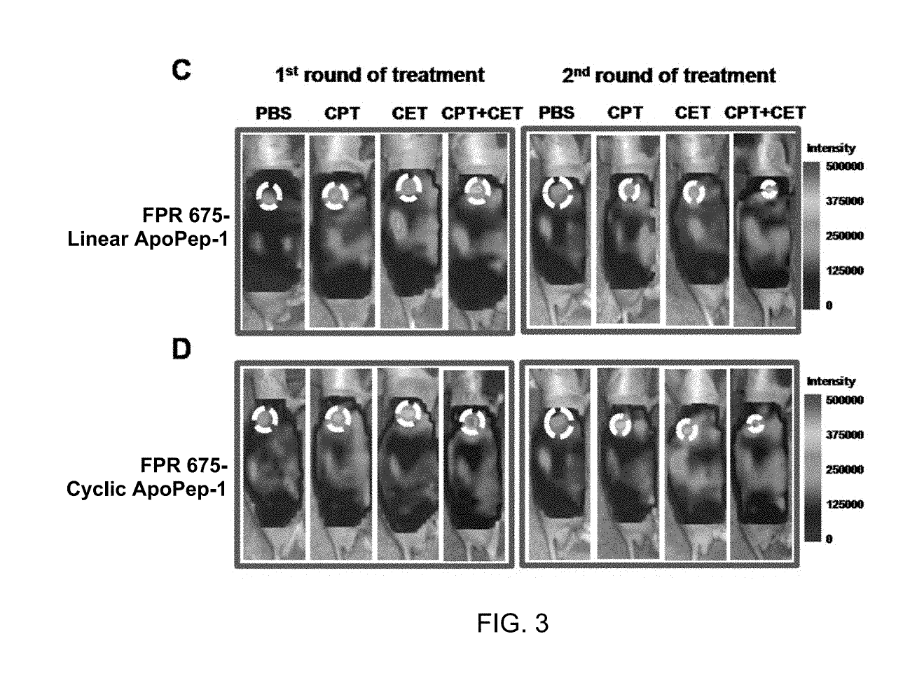

Representative whole body fluorescence images by linear and cyclic forms of ApoPep-1 are shown in panel C and panel D of FIG. 3, respectively). As shown in FIG. 3, the difference in fluorescence intensity between experimental groups was confirmed to be definitely differentiated by naked eyes in the fluorescence image by cyclic form of ApoPep-1 compared with linear form of ApoPep-1. Weak background fluorescence signals were observed in other organs, including the liver and lung (panel C and panel D of FIG. 3).

### Example 3

Measurement of Tumor Volumes and Weights after Anti-Tumor Treatment with Cisplatin and Cetuximab

To examine anti-tumor growth effect by cisplatin or cetuximab alone and in combination, tumor volumes and weights after the drug treatment were measured.

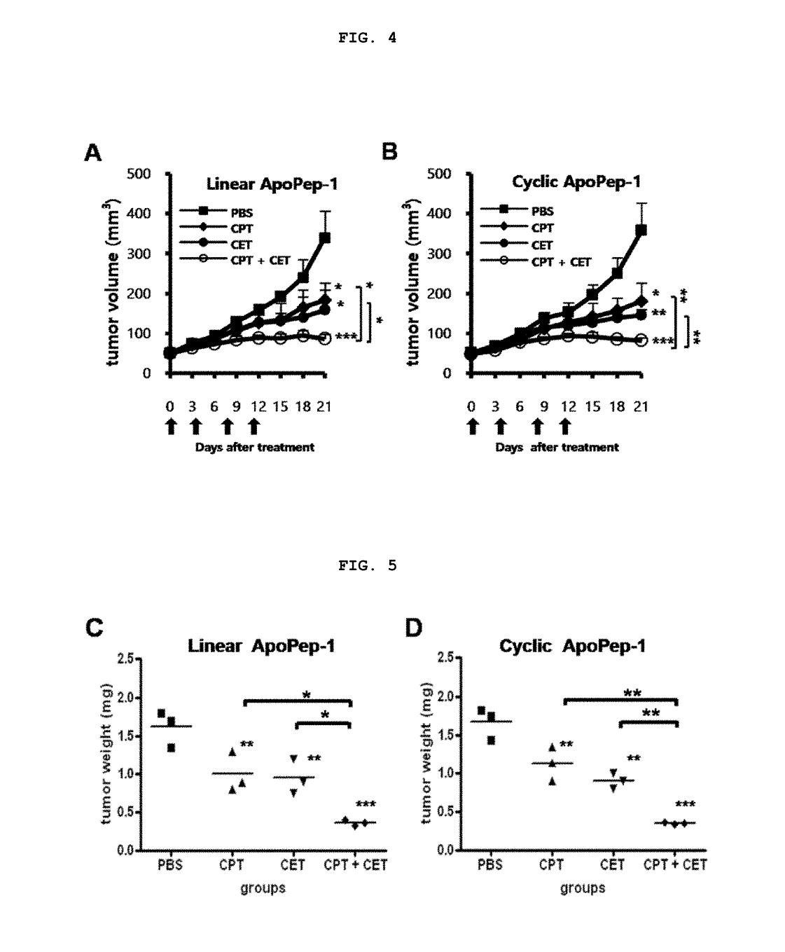

Treatment with cisplatin and cetuximab alone and in combination reduced tumor volumes compared with control group treated without drug, in the linear form of ApoPep-1 group (p<0.05, p<0.05, and p<0.001, sequentially, panel A of FIG. 4) and in the cyclic form of ApoPep-1 group (p<0.05, p<0.01, and p<0.001, sequentially, panel B of FIG. 4). Combined treatment of cisplatin and cetuximab reduced tumor volumes more efficiently, compared with treatment with cisplatin or cetuximab alone (p<0.05 and p<0.05, respectively, in the linear form of ApoPep-1 group, as shown in panel A of FIGS. 4; and p<0.01 and p<0.01, respectively, in the cyclic ApoPep-1 group, as shown in panel B of FIG. 4).

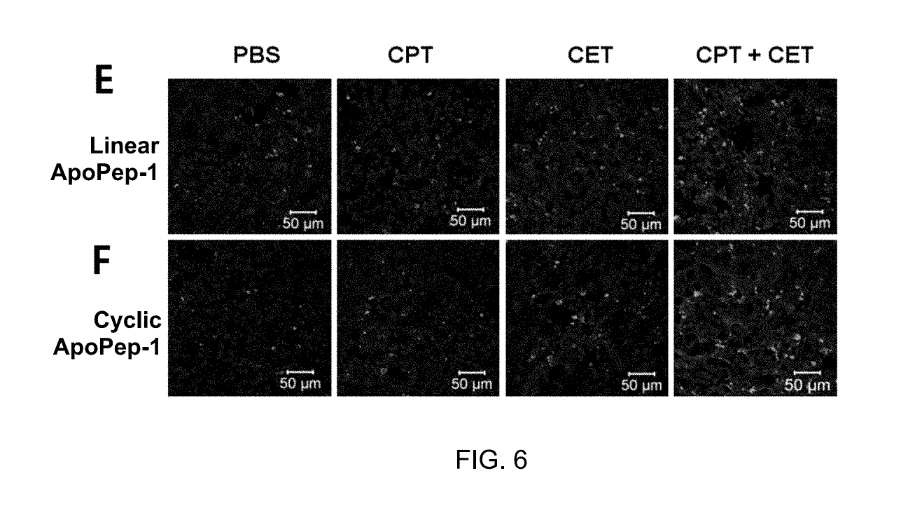

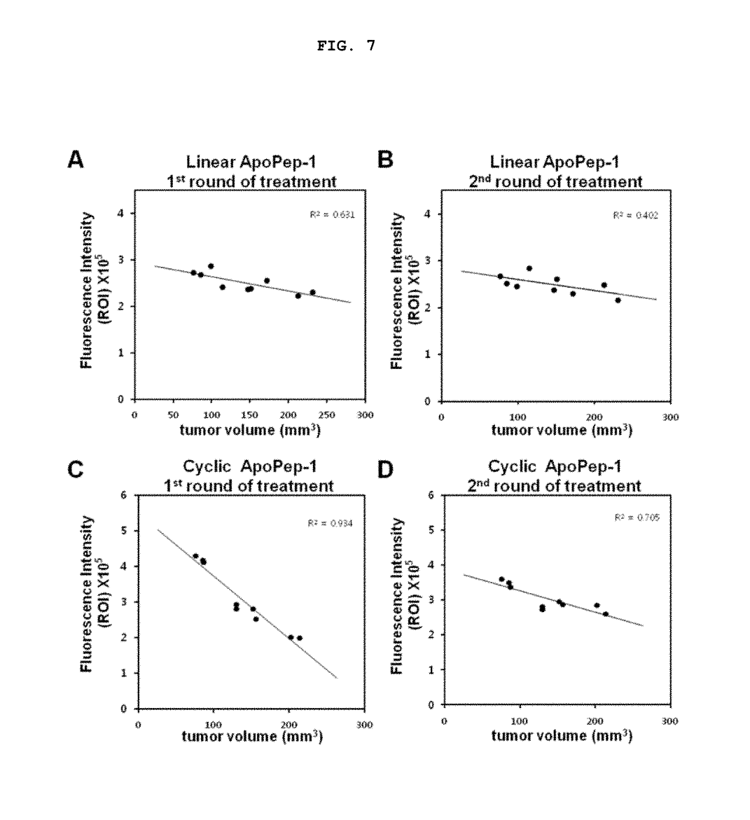

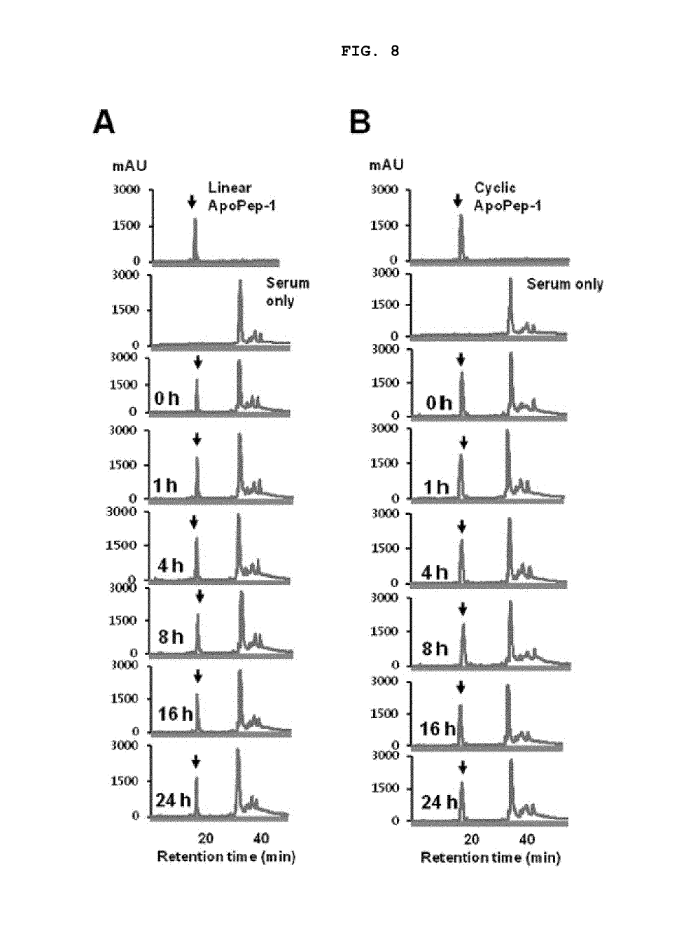

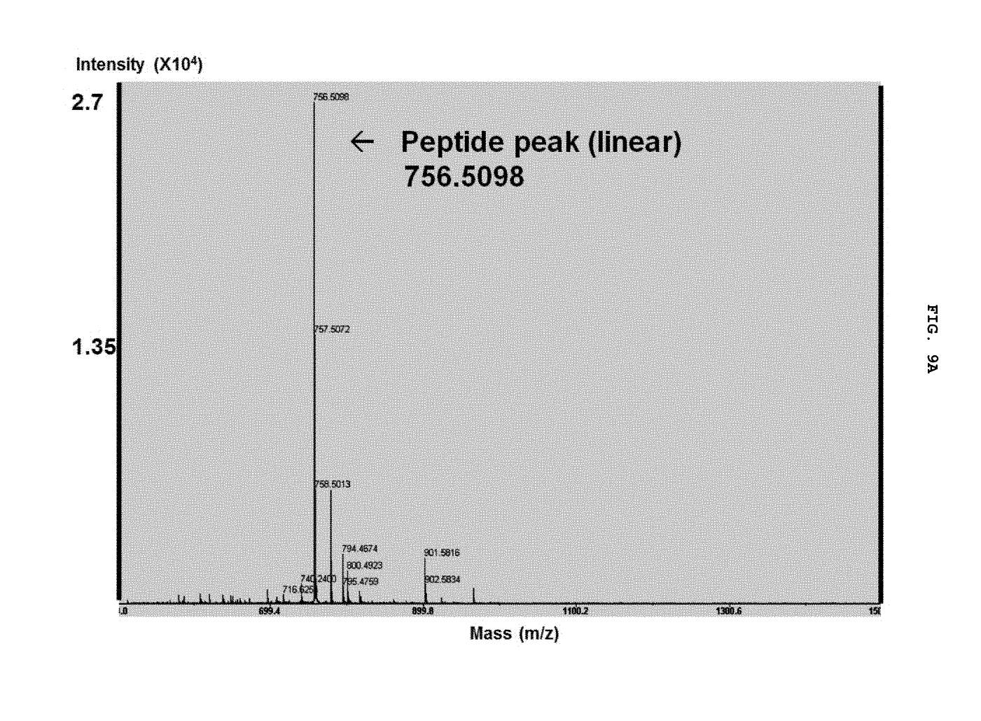

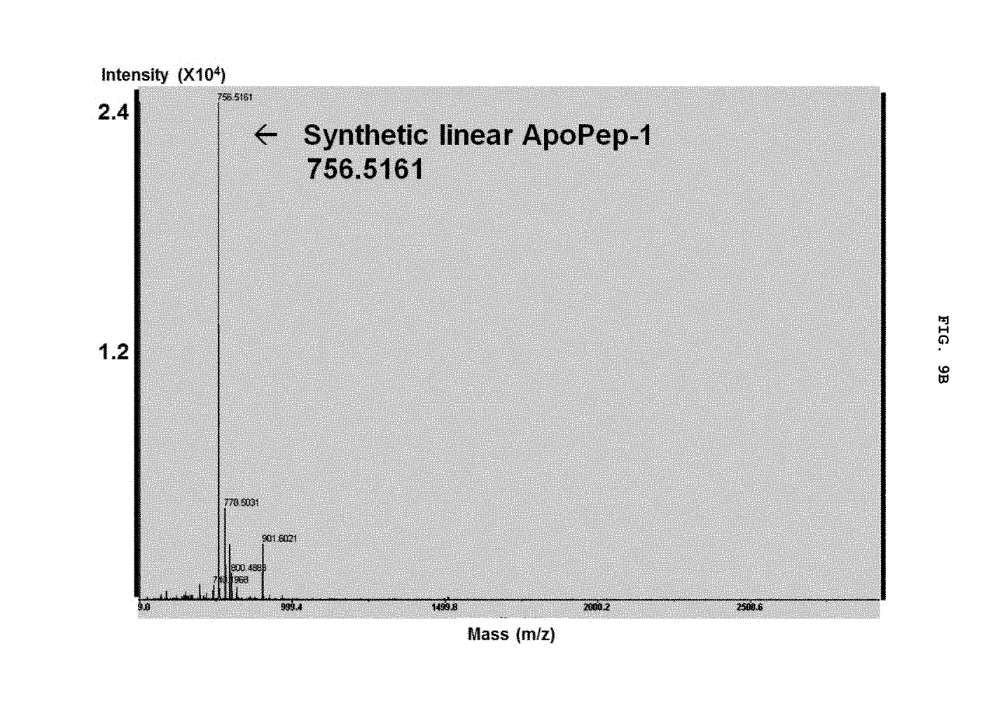

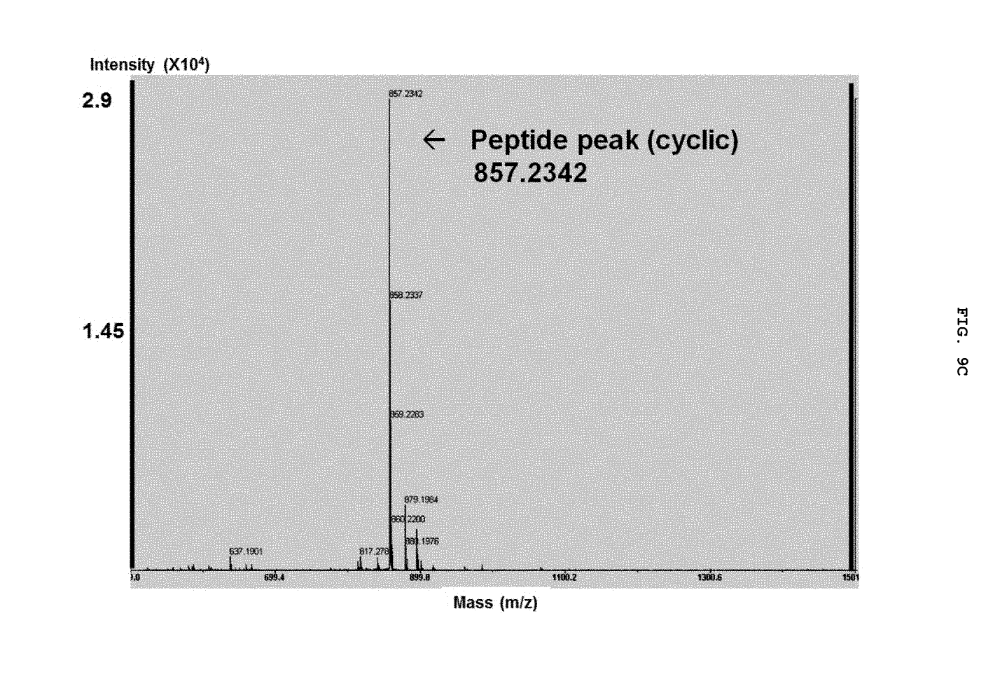

Also in changes in tumor weights after treatment with cisplatin and cetuximab alone and in combination, compared with untreated control, the above similar patterns were observed in the linear form of ApoPep-1 group (p<0.01, p<0.01, and p<0.001, sequentially, panel C of FIG. 5) and in the cyclic ApoPep-1 group (p<0.01, p<0.01, and p<0.001, sequentially, panel D of FIG. 5).

Combined treatment of cisplatin and cetuximab reduced tumor weights more efficiently, compared with treatment with cisplatin or cetuximab alone (p<0.05 and p<0.05, respectively, in the linear form of ApoPep-1 group, as shown in panel C of FIGS. 5; and p<0.01 and p<0.01, respectively, in the cyclic ApoPep-1 group, as shown in panel D of FIG. 5).

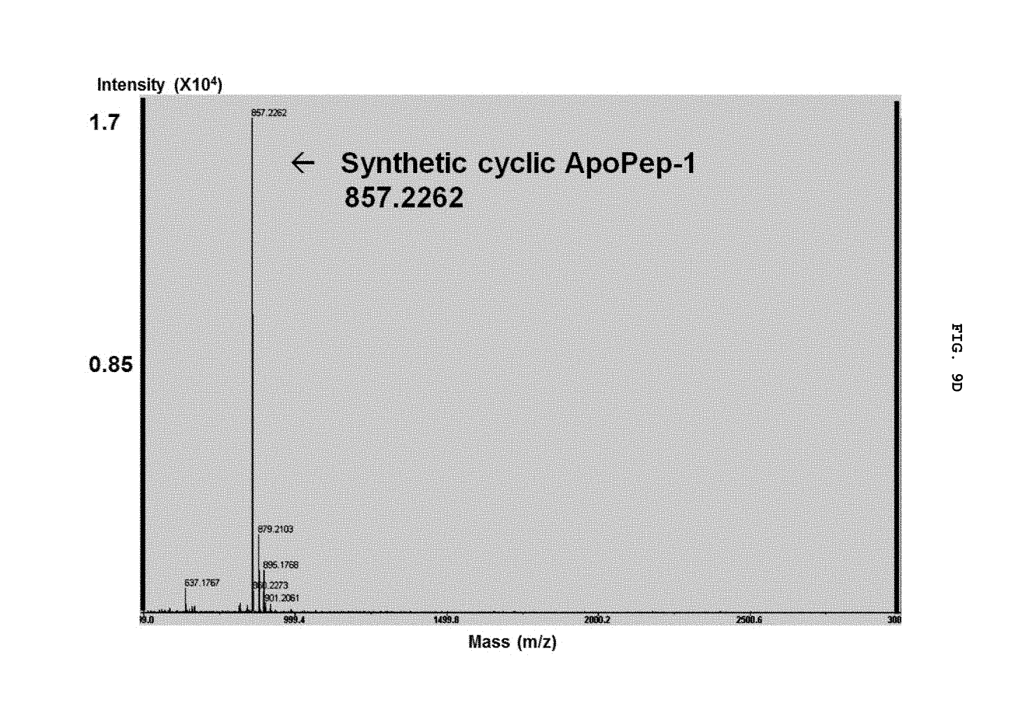

The levels of reduction in tumor volumes and weights after the treatment, between experimental groups injected with linear or cyclic form of ApoPep-1 were similar, and there were no differences in tumor volumes between those two groups at the time of imaging. As shown in FIG. 6, higher levels of apoptosis after treatment with cisplatin and cetuximab in combination, compared with treatment with cisplatin or cetuximab alone, was further demonstrated by the TUNEL staining of the tumor tissues (FIG. 6).

### Example 4

### Correlation Between Fluorescence Intensity and Tumor Size

After the first and second round of treatment, the correlation between the fluorescence intensity of in vivo imaging of apoptosis (measured by the same method at 1 week and 2 weeks after the initiation of treatment, respectively) and later-on tumor volume (at 3 weeks after the initiation of treatment). The fluorescence intensities of images taken by cyclic form of ApoPep-1 after the first round of treatment were inversely correlated with tumor volumes with the strongest agreement (correlation coefficient r²=0.934, panel C of FIG. 7). The above results showed high correlation, compared with the fluorescence intensities obtained by cyclic form of ApoPep-1 after the second round of treatment (r²=0.705, panel D of FIG. 7), the fluorescence intensities obtained by linear form of ApoPep-1 after the first round of treatment (r²=0.631, panel A of FIG. 7), and the fluorescence intensities obtained by linear form of ApoPep-1 after the second round of treatment (r²=0.402, panel B of FIG. 7). It can be seen through these results that the cyclic form of ApoPep-1 of the present invention can achieve fast diagnosis in tumor response to drugs in an initial stage (even about 1 week) after drug treatment.

### Example 5

Stability of Linear and Cyclic Forms of ApoPep-1 in the Serum

It was examined whether higher levels of imaging signals by the cyclic form of ApoPep-1 compared with those of the linear form of ApoPep-1 was due to the difference in serum stability of peptides. After incubation of the linear or cyclic form of ApoPep-1 with mouse serum up to 24 hours, the amount of the peptide remaining in the serum was analyzed. The peptide peak was separable from non-specific peaks of serum, and the amount of linear and cyclic forms of peptide remaining in the serum (as calculated by peak area) was not significantly changed up to 24 hours (panel A and panel B of FIG. 8, respectively). MS analysis of each peptide peak confirmed the identity of the linear (FIG. 9A) and cyclic (FIG. 9C) forms of ApoPep-1. These results suggest that both the linear and cyclic forms of ApoPep-1 are stable in the serum up to 24 hours with no difference in stability within the incubation time period, which means that the characteristics in which the cyclic form of ApoPep-1 peptide of the present invention shows significantly improved targeting activity compared with the linear form of peptide are not due to the difference in peptide stability in serum. Therefore, it was suggested that, in cyclic form of ApoPep-1 peptide of the present invention, an artificial structure that binds better to apoptotic cells (histone H1) is generated during the cyclization of the peptide.

### INDUSTRIAL APPLICABILITY

As set forth above, the present invention is directed to a cyclic peptide (cyclo[Cys-Gln-Arg-Pro-Pro-Arg-Cys] peptide) consisting of the amino acid sequence represented by SEQ ID NO: 2 and to a composition comprising the same as an active ingredient for detecting apoptotic cells, delivering a drug, and imaging.

However, the cyclic peptide (cyclo[Cys-Gln-Arg-Pro-Pro-Arg-Cys] peptide) of the present invention comprising the amino acid sequence represented by SEQ ID NO: 2 has an excellent effect of binding to (or targeting) apoptotic cells as compared with the corresponding linear peptide, thereby facilitating the detection of apoptotic cells and the in vivo imaging of the affected part undergoing apoptosis, while the detection and imaging signals exhibit a very high relevance in view of predicting the prognosis of a disease. Therefore, the cyclic peptide of the present invention can diagnose a response of a therapeutic drug to an abnormal cell proliferation-related disease at an early stage by binding with an imaging material, and can be used for the purpose of selectively delivering a drug to an apoptosis-relating disease tissue by conjugating with a therapeutic material. Accordingly, the present invention is highly industrially applicable.

1. A cyclic peptide consisting of the amino acid sequence of SEQ ID NO: 2 and specifically binding to apoptotic cells.

2. A composition for detecting apoptotic cells, the composition comprising the peptide of claim 1 as an active ingredient.

3. A composition for imaging an affected part by an apoptosis-related disease, the composition comprising the peptide of claim 1 as an active ingredient.

4. The composition of claim 3, wherein the apoptosis-related disease is any one selected from the group consisting of neoplastic disease, myocardial infarction, arteriosclerosis, neurodegenerative disease, and stroke.

5. The composition of claim 4, wherein the neoplastic disease is any one selected from the group consisting of brain cancer, neuroendocrine cancer, stomach cancer, lung cancer, breast cancer, ovarian cancer, liver cancer, bronchial cancer, nasopharyngeal cancer, laryngeal cancer, pancreatic cancer, bladder cancer, adrenal gland cancer, colorectal cancer, colon cancer, cervical cancer, prostate cancer, bone cancer, skin cancer, thyroid cancer, parathyroid cancer, and ureteral cancer.

6. The composition of claim 4, wherein the neurodegenerative disease is selected from the group consisting of Alzheimer's disease, Parkinson's disease, Huntington's disease, Amyotrophic lateral sclerosis, and Niemann-Pick disease.

7. A composition for screening an initial drug response of a test preparation having apoptosis-inducing activity in a subject afflicted with an abnormal cell proliferation-related disease, the composition comprising the peptide of claim 1 as an active ingredient.

8. The composition of claim 7, wherein the abnormal cell proliferation-related disease is a neoplastic disease or hyperproliferative vascular disease.

9. The composition of claim 2, wherein the peptide is labeled with any one selected from the group consisting of a chromogenic enzyme, a radioactive isotope, a chromophore, a luminescent material, a fluorescer, gadolinium, super paramagnetic particles, and ultrasuper paramagnetic particles.

10. A method for detecting apoptotic cells, the method comprising:
(a) mixing peptides of claim 1 with a sample;
(b) removing the peptides that are unbound or non-specifically bound; and
(c) determining a binding or non-binding of the peptides and a binding position of the peptides.

11. A method for screening an initial drug response of a test preparation in a subject afflicted with an abnormal cell proliferation-related disease, the method comprising:
(a) treating a target tissue of an affected part isolated from a subject with a test preparation having apoptosis-inducing activity, wherein the subject is afflicted with an abnormal cell proliferation-related disease;
(b) treating the test preparation-treated target tissue of step (a) and a control target tissue treated without a test preparation, with a peptide of claim 1 labeled with a labeling means; and
(c) detecting and comparing the labeling means in the peptide-treated target tissues in step (b).

12. The method of claim 11, further comprising (d) determining the target tissue as being responsive to the test preparation if an increased level of the labeling means is detected in the test preparation-treated target tissue in comparison with the control target tissue.

13. The method of claim 11, wherein the labeling means is any one labeling material selected from the group consisting of a chromogenic enzyme, a radioactive isotope, a chromophore, a luminescent material, a fluorescer, gadolinium, super paramagnetic particles, and ultrasuper paramagnetic particles.

14. A composition for delivering a drug for an apoptosis-related disease, the composition comprising the peptide of claim 1 as an active ingredient.

15. The composition of claim 14, wherein the apoptosis-related disease is any one selected from the group consisting of neoplastic diseases, myocardial infarction, arteriosclerosis, neurodegenerative diseases, and stroke.

16. A pharmaceutical composition for preventing and treating a neoplastic disease, the pharmaceutical composition comprising, as active ingredients, the peptide of claim 1 and an anti-tumor substance conjugated thereto.

17. The composition of claim 16, wherein the anti-tumor substance is conjugated to a drug selected from the group consisting of paclitaxel, doxorubicin, vincristine, daunorubicin, vinblastine, actinomycin-D, docetaxel, etoposide, teniposide, bisantrene, homoharringtonine, Gleevec (STI-571), cisplain, 5-fluouracil, adriamycin, methotrexate, busulfan, chlorambucil, cyclophosphamide, melphalan, nitrogen mustard, nitrosourea, streptokinase, urokinase, alteplase, angiotensin II inhibitor, aldosterone receptor inhibitor, erythropoietin, NMDA (N-methyl-d-aspartate) receptor inhibitor, lovastatin, rapamycin, Celebrex, Ticlopin, Marimastat, and Trocade.

18. A composition for preventing and treating a neurodegenerative disease, the composition comprising, as active ingredients, the peptide of claim 1 and a neurodegenerative disease therapeutic substance conjugated thereto.

19. A pharmaceutical composition for preventing and treating myocardial infarction, the pharmaceutical composition comprising, as active ingredients, the peptide of claim 1 and a myocardial infarction therapeutic substance conjugated thereto.

20. A pharmaceutical composition for preventing and treating arteriosclerosis, the pharmaceutical composition comprising, as active ingredients, the peptide of claim 1 and an arteriosclerosis therapeutic substance conjugated thereto.

21. A pharmaceutical composition for preventing and treating stroke, the pharmaceutical composition comprising, as active ingredients, the peptide of claim 1 and a stoke therapeutic substance conjugated thereto.

22. The composition of claim 14, wherein the composition further comprises any one labeling material selected from the group consisting of a chromogenic enzyme, a radioactive isotope, a chromophore, a luminescent material, a fluorescer, gadolinium, super paramagnetic particles, and ultrasuper paramagnetic particles.

23. A method for treating a neoplastic disease, the method comprising administering an effective amount of the peptide of claim 1 and an anti-tumor substance conjugated thereto to a subject in need thereof.

24. A method for preventing or treating a neurodegenerative disease, the method comprising administering an effective amount of the peptide of claim 1 and a neurodegenerative disease therapeutic substance conjugated thereto to a subject in need thereof.

25. A method for preventing or treating myocardial infarction, the method comprising administering an effective amount of the peptide of claim 1 and a myocardial infarction therapeutic substance conjugated thereto to a subject in need thereof.

26. A method for preventing or treating arteriosclerosis, the method comprising administering an effective amount of the peptide of claim 1 and an arteriosclerosis therapeutic substance conjugated thereto to a subject in need thereof.

27. A method for preventing or treating stroke, the method comprising administering an effective amount of the peptide of claim 1 and a stroke therapeutic substance conjugated thereto to a subject in need thereof.
